(19) United States
(12) Patent Application Publication
(10) Pub. No.: US 2020/0406227 A1
(43) Pub. Date: Dec. 31, 2020

(54) Carbon Skeleton Reinforced Porous Starch and Preparation Method thereof

(71) Applicant: Jiangnan University, Wuxi (CN)

(72) Inventors: Zhengyu JIN, Wuxi (CN); Enbo XU, Wuxi (CN); Zhengzong WU, Wuxi (CN); Aiquan JIAO, Wuxi (CN); Xing ZHOU, Wuxi (CN); Yaoqi TIAN, Wuxi (CN); Jinpeng WANG, Wuxi (CN); Yuxiang BAI, Wuxi (CN); Jianwei ZHAO, Wuxi (CN); Zhengjun XIE, Wuxi (CN); Xueming XU, Wuxi (CN); Na YANG, Wuxi (CN)

(73) Assignee: Jiangnan University, Wuxi (CN)

(21) Appl. No.: 16/475,126

(22) PCT Filed: Jul. 11, 2018
(86) PCT No.: PCT/CN2018/095255

§ 371 Date: Jul. 1, 2019

(30) Foreign Application Priority Data
Jun. 26, 2018 (CN) 201810670327.8

(51) Int. Cl. B01J 20/24; B01J 20/20; B01J 20/30; C12P 19/04; C12P 19/14; C12P 19/22

(52) U.S. Cl. CPC B01J 20/24; B01J 20/20; B01J 20/3085; C12P 19/22; C12P 19/04; C12P 19/14; B01J 20/3007

(57) ABSTRACT

A carbon skeleton reinforced porous starch and a preparation method thereof. The method of preparing the porous starch includes bonding starch to transition metal ions, then treating the starch attached with the transition metal ions and amylase via an extruding device, and finally forming a starch-based porous material having a high strength recombination structure, that is, the carbon skeleton reinforced porous starch.

### CROSS REFERENCE TO THE RELATED APPLICATIONS

This application is the national phase entry of International Application No. PCT/CN2018/095255, filed on Jul. 11, 2018, which claims priority from Chinese Patent Application 201810670327.8, filed on Jun. 26, 2018, the entire contents of which are incorporated herein by reference.

### TECHNICAL FIELD

The present disclosure relates to a starch-based porous material, and more particularly to a porous starch prepared by using a transition metal ion reinforced carbon skeleton.

### BACKGROUND

Porous starch is a new type of modified starch that changes the granular structure of the native starch by a biological, chemical or physical method to form a porous structure. Compared to the native starch, the porous starch has a greater porosity, a greater specific surface area, a lower bulk density and a better absorbability, and has been used widely in many fields, such as agriculture, medicine, environmental protection, food, papermaking, printing, detergents, and cosmetics.

At present, the biological method is the most commonly used, that is, starch is hydrolyzed with amylase to produce small pores on the partial surface thereof. However, the raw starch is less sensitive to enzyme at a temperature below the gelatinization temperature, while high temperature starch gelatinization destroys the supporting structure (i.e., skeletal structure) of the pore, therefore, the biological method is basically conducted at a low temperature, and an increased amount of enzyme or extended reaction time are often needed achieve higher yield. Moreover, the porous starch prepared by the method is subjected to a certain degree of damage to the overall structure, has poor physical and chemical properties, anti-dissolution as well as mechanical effect, has limitations such as functional simplex, often needs significant modification in the application, such as chemical cross-linking and surface functional group modification, and is laborious and time consuming.

### SUMMARY

### Technical Problem

In addition to the biological method, the porous starch can be prepared by a chemical method, such as acid hydrolysis, solvent exchange, and emulsion crosslinking. However, the method is complex in preparation process, large amounts of chemical reagents are introduced, and certain problems exist in the aspect of production cost and sustainable development. Furthermore, the starch treated with a chemical reagent is more fragile in structure than the porous starch prepared by the biological method, and therefore, the application thereof is limited.

The physical method has the least application, although many methods such as extrusion, ultrasonic, alcohol denaturing, spraying and mechanical impact are attempted, it is substantially difficult to form effective porous structures (in most cases, the surface is recessed and poor in uniformity), generally, the physical method is only used as a pre-treatment step to assist the preparation with the biological method.

### Technical Solution

In view of the above problems existing in the prior art, the applicant of the present disclosure provides a carbon skeleton reinforced porous starch and a preparation method thereof.

According to the method, the transition metal ion reinforced starch structure are utilized and the non-transition metal ion assisted enzyme effect functions stably, so that the physical extrusion-enzyme synergistic effect becomes an effective means of preparing the porous starch, and on the one hand, the enzyme dosage and the action time in the biological method are reduced to improve the mechanical strength of the product; on the other hand, the situation that the physical method can only be used as a pretreatment is changed to combine and simplify the original process.

The technical solutions of the present disclosure are described as follows:

A carbon skeleton reinforced porous starch, wherein the porous starch is prepared by the following method of: bonding starch to transition metal ions, then treating the starch attached with the transition metal ions and amylase via an extruding device, and finally forming a starch-based porous material having a high strength recombination structure, that is, the carbon skeleton reinforced porous starch.

The method of preparing the porous starch comprises the following specific steps:

(1) mixing the starch with a transition metal aqueous solution, stirring and soaking for 1-12 h under the condition of 20-40° C., pouring and sieving the starch, and rinsing for 1-3 times, and drying at 30-50° C. in an oven to obtain transition metal ion reinforced starch;

(2) mixing the amylase with a non-transition metal ion aqueous solution, stirring at 20-40° C., and mixing for 1-4 h to obtain a non-transition metal ion-amylase composite liquid;

(3) mixing and pre-moisturizing the transition metal ion reinforced starch obtained in the step (1) with the non-transition metal ion-amylase composite liquid obtained in the step (2) to obtain a premix, then feeding the premix into an extruding system and discharging to prepare the carbon skeleton reinforced porous starch.

The starch in the step (1) is one or more of corn starch, rice starch, potato starch and cassava starch; the transition metal is one or more of manganese, iron, cobalt, nickel, copper, zinc, zirconium and palladium added in a form of soluble metal salt.

The transition metal aqueous solution in the step (1) has an ion concentration of 0.1 to 5 mol/L; the mass to volume ratio of the starch to the transition metal aqueous solution is 50 to 250:1 g/L.

The amylase in the step (2) is one or more of mesophilic α-amylase, thermostable α-amylase, β-amylase and saccharifying enzyme; the non-transition metal ion is one or more of sodium, potassium, magnesium and calcium.

The non-transition metal ion aqueous solution in the step (2) has an ion concentration of 0.001 to 0.01 mol/L; the amylase and the non-transition metal ion aqueous solution has a mass to volume ratio of 0.2 to 2:1 g/L.

The transition metal ion reinforced starch and the non-transition metal ion-amylase composite liquid in the step (3) has a mass to volume ratio of 800-2500:1 g/L.

The premix in the step (3) has a water content of 28.5 to 55.5 wt %; the premix has an enzyme activity of more than 12 U/g, and after the enzyme activity is defined as the enzyme added per gram of starch, the enzyme activity of the system is more than 12 U.

The system parameters of the extrusion system in the step (3) are that: material temperature is 35-105° C., the pressure is less than 5 MPa, and the mechanical energy is less than 300 kJ/kg; the number of kneading or reversing element zones is less than or equal to two groups, the temperature from a feed port to a discharge port is set within a range of 30° C. to 100° C., a rotating speed of a screw is set at 50-250 rpm, and a die head is assembled or not assembled at the outlet.

### Advantageous Effect

The transition metal ions according to some embodiments of the present disclosure are absorbed into the surface or inside of the starch in an aqueous solution, and covalent binding occurs, thereby changing the physical and chemical properties, thermal properties and resistance to enzymatic hydrolysis of the starch. Since the binding of these transition metal ions to different portions of the starch is selective, the unbound region of the starch is relatively weakened. During the intense extrusion process, the relatively weakened portion of the starch can be rapidly degraded by the amylase to form a porous space, while the reinforced portion of the starch is can maintain a certain mixing and recombination structure, to support the porous space as a skeleton, and finally forms a “porous matrix”.

According to the method, the binding inhibition effect of certain transition metal ions on amylase is taken into account, the enzyme activity promoting ions can be introduced in advance into the active center of the amylase and then enter the extrusion process together. In terms of enzyme, an extruder barrel is a “high substrate” environment, these metal salts will release cations and protect the amylase during extrusion, so that the amylase can radiate out from the center to hydrolyze the starch substrate in the feedstock wrapped around (to avoid the reinforced portion of the starch), and gradually form a porous structure.

According to the method, the transition metal ion reinforced starch structure are utilized and the non-transition metal ion assisted enzyme effect functions stably, so that the physical extrusion-enzyme synergistic effect becomes an effective means of preparing the porous starch. Selection is made based on different raw materials, metal ions, enzyme preparations and operating conditions, the porous starch has a widespread aperture range (1-30 μm), a high strength in recombination structure, a strong water absorption/oil absorption capacity, and a good biodegradability; the extruder is used as a reaction vessel and the method has the advantages of continuity, solvent-free, product function, shape diversity and etc.

Compared to the prior art, some embodiments of the present disclosure combines the physical method for pretreatment and the biological method, the production steps are simple, the obtained porous starch has controllable average aperture range and overall size, a high structural strength, a good water absorption/oil absorption capacity, and a good biodegradability, moreover, the extruder is used as a reaction vessel and the method has the advantages of continuity, solvent-free, and sustainable production.

### DETAILED DESCRIPTION

The present disclosure will be specifically described with reference to examples.

### EXAMPLE 1

A carbon skeleton reinforced porous starch, wherein the preparation method includes the following specific steps:

(1) mixing 500 g of corn starch with 2 L, 5 mol/L of zirconium sulphate (Zr(SO₄)₂) aqueous solution, stirring and soaking for 12 h at 40° C., pouring and sieving the starch, and rinsing for 3 times, and drying at 50° C. in an oven to obtain zirconium ion reinforced starch;

(2) mixing 2 g of mesophilic α-amylase with 1 L, 0.01 mol/L of calcium chloride (CaCl₂) aqueous solution, stirring at 40° C., and mixing for 4 h to obtain a calcium-amylase composite liquid; and

(3) taking 450 g of zirconium ion enhanced starch (preparation loss rate is less than 3%, starch wet basis content 93.7%) to mix with 0.56 L of calcium-amylase composite liquid, pre-moisturizing to a water content of 55%, preparing an enzyme-containing premix, and then adding the moisturized enzyme-containing premix into a twin-screw extruder at a feeding rate of 2 kg/h. After being cooled and dried, the extrudate can be kept in an overall shape, and starch-based porous materials of different particle sizes also can be prepared by cutting (or crushing) and sieving. A kneading element and an adjacent reversing element are arranged in the extrusion slot of the extruder, a rotating speed of a screw is set at 250 rpm, the temperature at a feed port is set at 30° C., the intermediate temperature is 45° C., the temperature at a discharge port is set at 60° C., a die head with an aperture of 2 mm is assembled at the outlet, during the extrusion process, the pressure is 2.7 MPa and the mechanical energy is 145 kJ/kg.

The porous starch prepared in this embodiment has a pore size distribution range of 1-10 μm, a good water absorbability, structural strength and biodegradability, wherein, the water absorption rate can reach 446%, and the strength is 27.6 MPa.

### EXAMPLE 2

A carbon skeleton reinforced porous starch, wherein the preparation method includes the following specific steps:

(1) mixing 500 g of cassava starch with 10 L, 0.1 mol/L of manganese sulfate (MnSO₄) aqueous solution, stirring and soaking for 1 h at room temperature, pouring and sieving the starch, and rinsing once, and drying at 30° C. in an oven to obtain manganese ion reinforced starch;

(2) mixing 0.04 g of thermostable α-amylase with 0.2 L, 0.001 mol/L of potassium chloride (KCl) aqueous solution, stirring at 30° C., and mixing for 4 h to obtain a potassium-amylase composite liquid;

(3) taking 450 g of manganese ion enhanced starch (preparation loss rate is less than 3%, starch wet basis content 95.2%) to mix with 0.18 L of potassium-amylase composite liquid, pre-moisturizing to a water content of 29%, preparing an enzyme-containing premix, and then adding the moisturized enzyme-containing premix into a twin-screw extruder at a feeding rate of 2 kg/h. After being cooled and dried, the extrudate can be kept in an overall shape, and starch-based porous materials of different particle sizes also can be prepared by cutting (or crushing) and sieving.

A kneading element and no reversing element is arranged in the extrusion slot of the extruder, a rotating speed of a screw is set at 50 rpm, the temperature at a feed port is set at 50° C., the intermediate temperature is 60-80° C., the temperature at a discharge port is set at 95° C., a die head with an aperture of 2 mm is assembled at the outlet, during the extrusion process, material temperature is 56-103° C., the pressure is 4.8 MPa and the mechanical energy is 275 kJ/kg.

The porous starch prepared in this embodiment has a pore size distribution range of 5-20 μm, a good water absorbability, structural strength and biodegradability, wherein, the water absorption rate can reach 238%, and the strength is 19.2 MPa.

### EXAMPLE 3

A carbon skeleton reinforced porous starch, wherein the preparation method includes the following specific steps:

(1) mixing 500 g of rice starch with 5 L, 2.5 mol/L of ferric chloride (FeCl₃) aqueous solution, stirring and soaking for 6 h at 30° C., pouring and sieving the rice starch, and rinsing twice, and drying at 40° C. in an oven to obtain ferric ion reinforced starch;

(2) mixing 0.5 g of β-amylase with 0.5 L, 0.005 mol/L of sodium chloride (NaCl), stirring at 30° C., and mixing for 4 h to obtain a sodium-amylase composite liquid;

(3) taking 450 g of ferric ion enhanced starch (preparation loss rate is less than 3%, starch wet basis content 93.9%) to mix with 0.30 L of sodium-amylase composite liquid, pre-moisturizing to a water content of 43%, preparing an enzyme-containing premix, and then adding the moisturized enzyme-containing premix into a twin-screw extruder at a feeding rate of 2 kg/h. After being cooled and dried, the extrudate can be kept in an overall shape, and starch-based porous materials of different particle sizes also can be prepared by cutting (or crushing) and sieving.

Two kneading elements and no reversing element are arranged in the extrusion slot of the extruder, a rotating speed of a screw is set at 150 rpm, the temperature at a feed port is set at 40° C., the intermediate temperature is 40-50° C., the temperature at a discharge port is set at 60° C., a die head with an aperture of 2 mm is assembled at the outlet, during the extrusion process, material temperature is 42-63° C., the pressure is 3.4 MPa and the mechanical energy is 183 kJ/kg.

The porous starch prepared in this embodiment has a pore size distribution range of 10-30 μm, a good water absorbability, structural strength and biodegradability, wherein, the water absorption rate can reach 286%, and the strength is 26.5 MPa.

1. A carbon skeleton reinforced porous starch, wherein the carbon skeleton reinforced porous starch is prepared by the following method:
bonding starch to transition metal ions, then treating the starch attached with the transition metal ions and amylase via an extruding device, and finally forming the carbon skeleton reinforced porous starch.

2. The carbon skeleton reinforced porous starch according to claim 1, wherein the method of preparing the carbon skeleton reinforced porous starch comprises the following steps:
(1) mixing the starch with a transition metal aqueous solution, stirring and soaking for 1-12 h under a condition of 20-40° C., pouring and sieving the starch, and rinsing for 1-3 times, and drying at 30-50° C. in an oven to obtain a transition metal ion reinforced starch;
(2) mixing the amylase with a non-transition metal ion aqueous solution, stirring at 20-40° C., and mixing for 1-4 h to obtain a non-transition metal ion-amylase composite liquid; and
(3) mixing and pre-moisturizing the transition metal ion reinforced starch obtained in the step (1) with the non-transition metal ion-amylase composite liquid obtained in the step (2) to obtain a premix, then feeding the premix into the extruding device and discharging to form the carbon skeleton reinforced porous starch.

3. The carbon skeleton reinforced porous starch according to claim 2, wherein the starch in the step (1) is one or more selected from the group consisting of a corn starch, a rice starch, a potato starch and a cassava starch; the transition metal is one or more selected from the group consisting of manganese, iron, cobalt, nickel, copper, zinc, zirconium and palladium added in a form of a soluble metal salt.

4. The carbon skeleton reinforced porous starch according to claim 2, wherein the transition metal aqueous solution in the step (1) has an ion concentration of 0.1 to 5 mol/L; a mass to volume ratio of the starch to the transition metal aqueous solution is 50 to 250:1 g/L.

5. The carbon skeleton reinforced porous starch according to claim 2, wherein the amylase in the step (2) is one or more selected from the group consisting of mesophilic α-amylase, thermostable α-amylase, β-amylase and saccharifying enzyme; the non-transition metal ion is one or more selected from the group consisting of sodium, potassium, magnesium and calcium.

6. The carbon skeleton reinforced porous starch according to claim 2, wherein the non-transition metal ion aqueous solution in the step (2) has an ion concentration of 0.001 to 0.01 mol/L; the amylase and the non-transition metal ion aqueous solution has a mass to volume ratio of 0.2 to 2:1 g/L.

7. The carbon skeleton reinforced porous starch according to claim 2, wherein the transition metal ion reinforced starch and the non-transition metal ion-amylase composite liquid in the step (3) has a mass to volume ratio of 800-2500:1 g/L.

8. The carbon skeleton reinforced porous starch according to claim 2, wherein the premix in the step (3) has a water content of 28.5 to 55.5 wt %; the premix has an enzyme activity of more than 12 U/g, and after an enzyme activity is defined as the enzyme added per gram of starch, the enzyme activity of the system is more than 12 U.

9. The carbon skeleton reinforced porous starch according to claim 2, wherein system parameters of the extruding device in the step (3) include a material temperature is 35-105° C., a pressure is less than 5 MPa, and a mechanical energy is less than 300 kJ/kg; number of kneading or reversing element zones are less than or equal to two groups, a temperature from a feed port to a discharge port is set within a range of 30° C. to 100° C., a rotating speed of a screw is set at 50-250 rpm, and a die head is assembled or not assembled at an outlet.
